(19) United States
(12) Patent Application Publication
(10) Pub. No.: US 2017/0238836 A1
(43) Pub. Date: Aug. 24, 2017

(54) SYSTEM AND METHOD FOR DATA RECONSTRUCTION IN SOFT-FIELD TOMOGRAPHY

(71) Applicant: General Electric Company, Schenectady, NY (US)

(72) Inventors: Kimmo Henrik Uutela, Helsinki (FI); Matti Lassas, Helsinki (FI); Petri Ola, Helsinki (FI); SamuIi Siltanen, Helsinki (FI); Ville Kolehmainen, Kuopio (FI); Alexander Seth Ross, Albany, NY (US)

(73) Assignee: General Electric Company, Schenectady, NY (US)

(21) Appl. No.: 15/419,450

(22) Filed: Jan. 30, 2017

Related U.S. Application Data
(63) Continuation of application No. 13/273,874, filed on Oct. 14, 2011.

(51) Int. Cl. A61B 5/053; G06T 11/00; G06T 7/00; A61B 5/00

(52) U.S. Cl. CPC A61B 5/0536; A61B 5/7257; G06T 11/008; G06T 7/0012; A61B 2562/0209; A61B 2562/04; A61B 2576/00; G06T 2207/10072

(57) ABSTRACT

A soft-field tomography system includes a plurality of transducers, one or more excitation drivers, one or more response detectors, and a soft-field reconstruction module. The transducers are configured for positioning proximate a surface of an object to be imaged, and are configured to apply excitations to the surface of the object. The excitation drivers are coupled to the transducers and configured to generate excitation signals to be applied by the transducers. The response detectors are coupled to the transducers and configured to measure a response of the object at the transducers to acquire an Electrical Impedance Tomography (EIT) data set. The soft-field reconstruction module is configured to select a model domain for the EIT data set that represents a predetermined shape, determine a minimally anisotropic error in the model domain, and perform isotropization using the determined minimally anisotropic error to recover a boundary shape and isotropic conductivity.

### RELATED APPLICATION

This application is a continuation of U.S. patent application Ser. No. 13/273,874, filed Oct. 14, 2011, entitled “SYSTEM AND METHOD FOR DATA RECONSTRUCTION IN SOFT-FIELD TOMOGRAPHY,” the entire subject matter of which is incorporated herein.

### BACKGROUND

The subject matter disclosed herein relates generally to data reconstruction systems and methods, and more particularly to systems and methods to reconstruct data in soft-field tomography.

Soft-field tomography, such as Electrical Impedance Tomography (EIT) (also referred to as Electrical Impedance Spectroscopy (EIS)), diffuse optical tomography, elastography, and related modalities may be used to measure the internal properties of an object, such as the electrical properties of materials comprising internal structures of an object (e.g., a region of a human body). For example, in EIT systems, an estimate is made of the distribution of electrical conductivities of the internal structures. Such EIT systems reconstruct the conductivity and/or permittivity of the materials within the area or volume based on an applied excitation (e.g., current) and a measured response (e.g., voltage) typically acquired at a surface of the area or volume by a number of transducers. For example, electrodes may be attached to the skin of a patient, which are used to perform EIT data acquisition. Visual distributions or images of the estimates can then be formed.

In EIT, the complex conductivity distributions within a volume are determined using assumed known applied electrical excitations, apriori geometry and surface electrode data, and signal measurement data from transducers coupled to the volume under test. An electromagnetic model with assumptions about the volume and electrode geometry, boundary conditions, the applied excitation, and the interior conductivity distribution are then used to determine a predicted response to a given excitation. The inverse problem in EIT is to determine the spatial distribution of complex conductivities that give rise to the measured data.

For example, the shape of the boundary of the body is conventionally assumed to be known in the computation of the conductivity image. However, that assumption is usually not practical. In particular, when monitoring heart and lung function of an unconscious intensive care patient using EIT, the measurement electrodes are attached around the chest on the skin of the patient. The shape of a cross-section of the chest is not a perfect cylinder. Further, even if the shape of the cross-section is obtained from another imaging modality, the shape of the thorax varies as the patient changes position and breathes, and therefore the boundary shape obtained from other available measurements would be approximate at the best. Also, the electrodes often have to be attached quickly, and it is not always possible to attach the electrodes systematically or record exact positions accurately. These issues lead to the potential for serious artifacts due to the boundary shape or the position of the electrodes being incorrectly modeled in the computation.

Some methods, such as difference imaging have been used in an attempt to overcome the problem of inaccurately known body shape or electrode locations. However, this approach is highly approximate as the actual nonlinear forward mapping is approximated by a linear mapping. Further, reconstructing conductivity differences based on differences of measurements reduces the effect of inaccurately known geometry only to a certain extent. Moreover, breathing artifacts are still present in the reconstructions. In some applications, such as when monitoring the lungs, difference imaging may be insufficient for the detection of clinically relevant conditions.

### BRIEF DESCRIPTION

In accordance with an embodiment, a method for Electrical Impedance Tomography (EIT) data reconstruction is provided. The method includes selecting a model domain for an EIT data set, determining a minimally anisotropic error in the model domain and correcting the model domain. The method also includes performing isotropization using the determined minimally anisotropic error to recover a boundary shape and isotropic conductivity for the EIT data set.

In accordance with another embodiment, a method for recovering boundary shape and electrode position in Electrical Impedance Tomography (EIT) data reconstruction is provided. The method includes determining a minimally anisotropic error using a set of parameters that define one or more coordinates or a coordinate transformation and correcting an EIT model using the determined minimally anisotropic error. The method also includes recovering a boundary shape and electrode position of an object with the corrected EIT model.

In accordance with yet another embodiment, a soft-field tomography system is provided that includes a plurality of transducers configured for positioning proximate a surface of an object and one or more excitation drivers coupled to the plurality of transducers and configured to generate excitation signals for the plurality of transducers. The soft-field tomography system also includes one or more response detectors coupled to the plurality of transducers and configured to measure a response of the object at the plurality of transducers to the excitation applied by the plurality of transducers based on the excitation signals. The soft-field tomography system further includes a soft-field reconstruction module configured to reconstruct a property distribution based on the excitation signals and the measured response using a determined minimally anisotropic error to correct a model domain for the object and perform isotropization using the determined minimally anisotropic error to recover a boundary shape and isotropic conductivity for the EIT data set.

### BRIEF DESCRIPTION OF THE DRAWINGS

The presently disclosed subject matter will be better understood from reading the following description of non-limiting embodiments, with reference to the attached drawings, wherein below:

FIG. 1 is a simplified block diagram illustrating a soft-field tomography system formed in accordance with various embodiments.

FIG. 2 is a simplified diagram illustrating reconstruction of a property distribution.

FIG. 3 is a diagram illustrating transducers in accordance with various embodiments.

FIG. 4 is a block diagram illustrating soft-field tomography information flow in accordance with various embodiments.

FIG. 5 is a flowchart of a method for recovering boundary shape and conductivity in Electrical Impedance Tomograhy (EIT) in accordance with various embodiments.

FIG. 6 are images illustrating EIT reconstruction.

FIG. 7 are images illustrating boundary recovery in accordance with various embodiments.

FIG. 8 is a diagram illustrating EIT reconstruction with correction for electrode placement in accordance with various embodiments.

### DETAILED DESCRIPTION

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers, circuits or memories) may be implemented in a single piece of hardware or multiple pieces of hardware. It should be understood that the various embodiments are not limited to the arrangements, component/element interconnections and instrumentality shown in the drawings.

As used herein, a module or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” a module or a plurality of modules having a particular property may include additional such modules not having that property.

Various embodiments provide systems and methods for soft-field data acquisition and reconstruction, and in particular to body shape and electrode location correction in soft-field tomography, especially Electrical Impedance Tomography (EIT) (also referred to as Electrical Impedance Spectroscopy (EIS)). A technical effect of at least one embodiment is the recovery of boundary shape and electrode position from EIT data.

It should be noted that although the various embodiments are described herein in connection with EIT, the various embodiments may be implemented in connection with other soft-field tomography systems, such as Diffuse Optical Tomography (DOT), Near InfraRed Spectroscopy (NIRS), thermography, elastography or microwave tomography, and related modalities. It also should be noted that as used herein, “soft-field tomography” refers generally to any tomographic or multidimensional extension of a tomographic method that is not “hard-field tomography”.

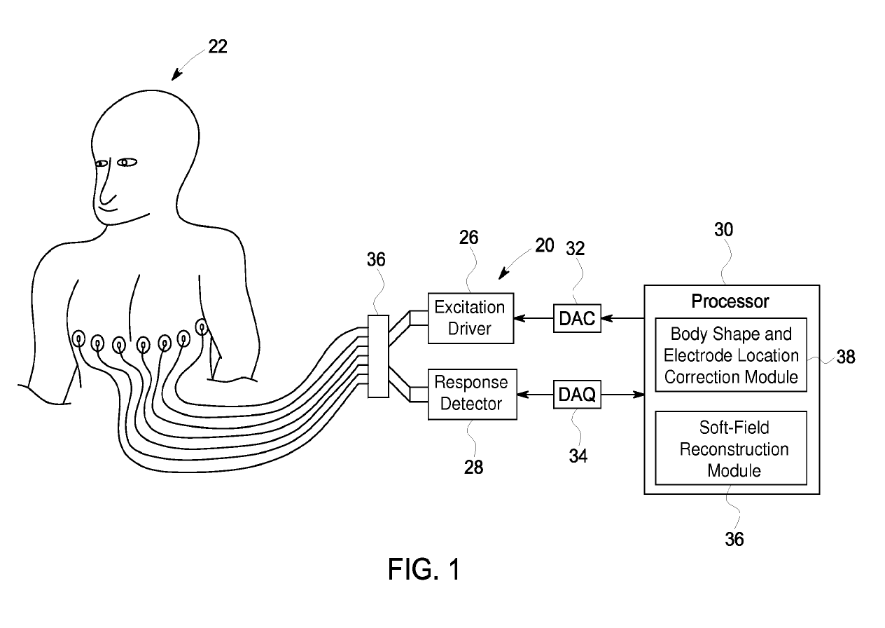

One embodiment of a soft-field tomography system 20 is illustrated in FIG. 1, which may be a transducer-based system, for example, an electrode-based system, such as a patient monitor that may form part of an electrocardiography (ECG) monitoring device or an impedance cardiography module. However, the soft-field tomography system 20 may also be an EIS/EIT system or other separate unit. The soft-field tomography system 20 may be used to obtain electrical impedance measurements of an object, illustrated as a patient 22. For example, electrical impedance measurements obtained may be used to monitor heart and lung function of an unconscious intensive care patient. In some embodiments, patient monitoring or tissue characterization generally may be provided.

In the illustrated embodiment, the soft-field tomography system 20 includes a plurality of transducers 24, which are shown as a plurality of electrodes, positioned at or proximate a surface of the patient 22. In a healthcare application (e.g., patient monitoring) the plurality of the transducers 24 are attached to the skin of the patient 22. It should be noted that although seven transducers 24 are illustrated, more or fewer transducers 24 may be used. For example, the transducers 24 extend around the chest of the patient 22 in the illustrated embodiment.

In other applications, the transducers 24 may be positioned at a surface of the object (e.g. electrodes, thermal sources, ultrasound transducers), near the surface of the object (e.g., radiofrequency antenna), or penetrating the surface of the object (e.g., needle electrodes). Thus, the transducers 24 may take different forms, such as surface-contacting electrodes, standoff electrodes, capacitively coupled electrodes, conducting coils, and antennas, among others.

It should be noted that other types of transducers may be used to generate different types of excitations, for example, in addition to current, other sources of excitation include voltage, magnetic fields or radio-frequency waves, among others. Thus, the transducers also may be surface-contacting electrodes, standoff electrodes, antennas, and coils, among others.

The soft-field tomography system 20 is used to determine the electrical properties of materials within the patient 22. For example, the spatial distribution of electrical conductivity (a) and/or permittivity (s) may be determined inside the patient 22 or other volume. Thus, internal properties of the patient 22 (e.g., a patient) may be determined.

An excitation driver 26 and a response detector 28 are coupled to one or more of the transducers 24, and are each connected to a processor 30 (e.g., a computing device), which may include other components therebetween. In one embodiment, the excitation driver 26 and the response detector 28 are physically separate devices. In other embodiments, the excitation driver 26 and the response detector 28 are physically integrated as one module. It should be noted that an excitation driver 26 may be provided in connection with at least one of the transducers 24. The processor 30 sends instructions to the excitation driver 26 through a digital to analog converter (DAC) module 32 that drives the transducers 24 and receives data from the response detector 28 through a data-acquisition element (DAQ) module 34. It should be noted that an excitation driver 26 may be provided in connection with all of the transducers 24 or a subset of the transducers 24.

In the illustrated embodiment, the region of interest is the human body region, such as a head, a chest, or a leg, wherein, air, blood, fat, muscle, and other tissues have different electrical conductivities. An electrical impedance distribution generated by the soft field tomography system 20 shows conditions of the internal properties (e.g., material properties) of the human body region, and thus can assist in monitoring the patient 22, as well as for the diagnoses of diseases, for example, associated with hemorrhage, tumor, and lung function, among others. In other embodiments, the soft field tomography system 20 can be used for generating a visual representation of the electrical impedance distribution in a variety of other applications, such as for determining the material properties in a mixed flow including oil and water, or soil analysis for mine exploration, among others. Thus, the object is not limited to the patient 22 and any object, such as animals or non-living objects are also subject to the techniques detailed herein.

In various embodiments, the transducers 24 are formed from any suitable material used to establish an excitation (e.g., EIS compatible electrical current). For example, the transducers 24 may be formed from one or more metals such as copper, gold, platinum, steel, silver, and alloys thereof. Other exemplary materials for forming the transducers 24 include non-metals that are electrically conductive, such as a silicon based materials used in combination with micro-circuits. In the embodiment where the object is a human body region, namely the patient 22, the transducers 24 may be formed from silver-chloride. Additionally, the transducers 24 may be formed in different shapes and/or sizes, for example, as rod-shaped, flat plate-shaped, or needle-shaped structures. It should be noted that in some embodiments, the transducers 24 are insulated from one another. In other embodiments, the transducers 24 can be positioned in direct ohmic contact with the object 22 or capacitively coupled to the object 22.

In various embodiments, a processor 30 is provided that includes a soft-field reconstruction module 36. The soft-field reconstruction module 36 may form part of or be any type of processor or computing device that performs soft-field reconstruction based at least in part on received responses from the transducers 24. Additionally, a body shape and electrode location correction module 38 is also provided that performs body shape recovery and correction for electrode position placement as described in more detail herein.

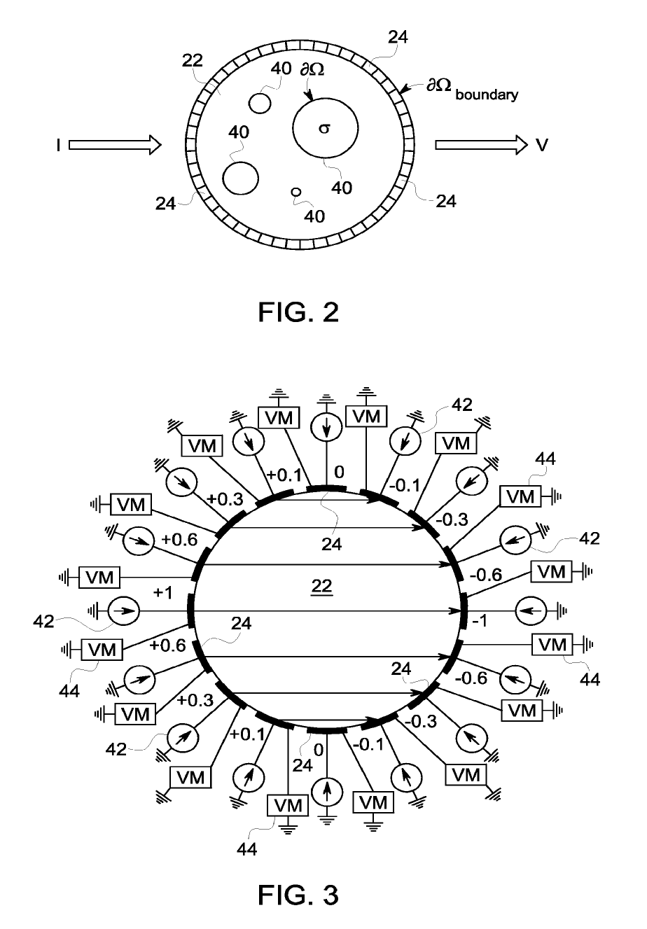

As illustrated in FIG. 2, a soft-field reconstruction is performed to identify ROIs 40 (e.g., blood in the brain) within the object 22. As shown, the response detector 28 (shown in FIG. 1) measures a response voltage (or a response current) on the transducers 24 in response to the current (or voltage) applied by the excitation driver 26 (shown in FIG. 1) to the transducers 24.

It should be noted that the response detector 28 also may include one or more analog-signal-conditioning elements (not shown) that amplifies and/or filters the measured response voltage or current. In other embodiments, the processor 30 of the soft-field tomography system 20 includes a signal conditioning element for amplifying and/or filtering the response voltage or response current received from the response detector 28.

Thus, as illustrated in FIG. 3, the excitation driver 26 applies an excitation pattern on the geometry by applying a load current 42 on each of the transducers 24. The response detector 28 is illustrated as having a plurality of voltage measuring devices, such as voltmeters 44, for measuring a voltage at the surface of the patient 22 at the transducers 24. It should be noted that the excitation pattern and measured response are simplified for illustration and the excitation and conductivity distribution may be more complex. Additionally, the illustrated values are provided for simplicity and ease of understanding.

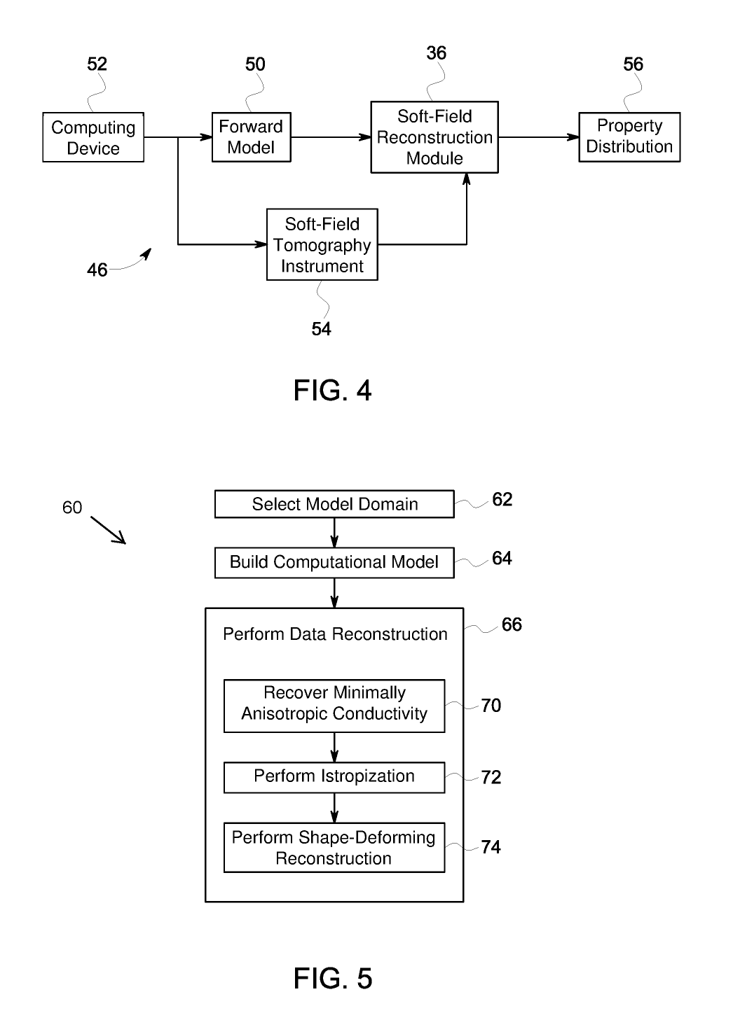

The soft-field reconstruction module 30, thus, computes a response of the patient 22 to the applied excitation. For example, an EIS information flow 46 is illustrated in FIG. 4. In particular, a forward model 50 is used based on excitations from a computing device 52, to predict voltages (predicted data), which are provided to the soft-field reconstruction module 36. In one embodiment, an inverse problem relating the measured responses (e.g., measured signals), the applied excitations, and the electrical conductivity distribution inside of the patient 22 being tested or interrogated by the soft-field tomography system 20 is solved by the reconstruction module 36 using any suitable EIS reconstruction technique.

The excitations are applied to the patient 22 (shown in FIGS. 1 and 3) by a soft-field tomography instrument 54, which may include the transducers 24 and other excitation and measurement components, and thereafter measured voltages (measured data) are communicated to the reconstruction module 36. The soft-field reconstruction module 30 then performs reconstruction, which includes performing body shape recovery and electrode location correction in accordance with various embodiments, to generate an estimate of a property distribution 56, for example, an impedance distribution, to identify the ROIs 40 within the patient 22. It should be noted that the various components may be physically separate components or elements may be combined. For example, the soft-field reconstruction module 36 may form part of the soft-field tomography system 20 (as illustrated in FIG. 1).

Using various embodiments, soft-field reconstruction with body shape recovery and electrode location correction is provided. The various embodiments determine an anisotropic error to provide correction for the model used for the reconstruction, such as the forward model 50. Thus, in various embodiments the model used for BIT reconstruction is modified or tuned using a set of parameters that corrects for assumptions regarding the shape of the object and the electrode location, namely the location of the electrodes on the object. Accordingly, various embodiments provide a set of equations to recover the shape of the object in the model domain.

A method 60 as shown in FIG. 5 is provided in one embodiment. The method 60 recovers boundary shape (and electrode location) and conductivity in EIT. The method 60 will first be described generally followed by a more detailed description. More particularly, the method 60 is performed to (i) recover an unknown isotropic conductivity γ in a measurement domain Ω and (ii) approximate the unknown shape of the boundary δΩ from discrete current-to-voltage measurements using N electrodes (as described herein) on the boundary δΩ. It should be noted that in some embodiments the data is a finite matrix that approximates the Robin-to-Neumann map R.

In one embodiment, a model domain is selected at 62 to provide an approximation Ωₘ to the actual domain Ω. For example, in a medical application when motioning a patient, Ωₘ may be selected as a disc having approximately the same area as Ω. It should be noted that the model domain may include, for example, the model of the shape of a conductor or electrode and/or the electrode positions. Thereafter, at 64, a computational model is provided. For example, in one embodiment, a computational model for discrete Robin-to-Neumann data from a given (possibly anisotropic) conductivity in Ωₘ measured using J electrodes on δΩₘ is formed.

Data reconstruction is then performed at 66. The data reconstruction generally includes recovering a minimally anisotropic conductivity at 70. In one embodiment, the recovery includes computing γₑ(x), which is the least anisotropic of all of the conductivities in the model domain Ωₘ and that results in the same data matrix that was measured on δΩ. The function may be defined as follows:

η(x):=det(γₑ(x))^{1/2} Eq. 1

The function defined by Equation 1 results in a distorted image inside Ωₘ of the original isotropic conductivity.

Isotropization is then performed at 72. In one embodiment, the isotropization includes determining numerically isothermal coordinates z→Fᵢ(z) corresponding to γₑ(x). The result of the isotropization is an approximate recovery of the domain deformation and boundary shape.

A shape-deforming reconstruction is then performed at 74. In one embodiment, the isothermal coordinates are used to reconstruct the original isotropic conductivity approximately as follows:

η(Re(Fᵢ(z)),Im(Fᵢ(z))) Eq. 2

With respect to recovering a minimally anisotropic conductivity at 70, quasiconformal maps are used. In general, the following are open sets:

Ω,\tilde{Ω}

[symbol]

[symbol]

An orientation-preserving homeomorphism F: Ω→\tilde{Ω} is called K-quasiconformal is defined as:

$$D ( z ) \leq K for a . e . z ∈ Ω , where D ( z ) = \frac{\vert \partial F ( z ) \vert + \vert \overline{\partial } F ( z ) \vert}{\vert \partial F ( z ) \vert - \vert \overline{\partial } F ( z ) \vert} \geq 1 \qquad Eq . 3$$

In Equation 3

$$\overline{\partial } = \frac{1}{2} ( \frac{\partial }{\partial {x}_{1}} + i \frac{\partial }{\partial {x}_{2}} ) , \partial = \frac{1}{2} ( \frac{\partial }{\partial {x}_{1}} - i \frac{\partial }{\partial {x}_{2}} )$$

and the derivatives may be classical or weak derivatives

There is also a geometric definition of a quasiconformal maps. It should be noted that conformal maps take infinitesimal disks at z to infinitesimal disks at f(z), and the radii are dilated by |f′(z)|. More generally, a homeomorphism f is quasiconformal on a domain Ω if infinitesimal disks at any zε68 Ω get mapped to infinitesimal ellipses at f(z).

The ratio of the larger semi-axis to the smaller semi-axis is the dilation D(z) of f at z, and taking the supremum over zεΩ yields a maximal dilation. This dilation of infinitesimal disks causes isotropic conductivities change to anisotropic conductivities in push-forwards with quasiconformal maps.

The inaccurately known boundary in various embodiments is considered as the boundary of the deformed model domain. This deformation corresponds to a sufficiently smooth diffeomorphism F that maps the original measurement domain Ω to another domain \tilde{Ω}. Then, if f=F|_{δΩ} with u solving the following with a Robin boundary value h, ũ=u∘F⁻¹ and \tilde{h}(x)=h(f⁻¹(x)):

∇·γ∇μ=0 in Ω Eq. 4

Then, ũ solves the conductivity equation:

∇·\tilde{γ}∇\tilde{μ}=0, in \tilde{Ω},

\tilde{z}v·\tilde{γ}∇\tilde{μ}+\tilde{μ}|_{∂\tilde{Ω}}=h̅, Eqs. 5 and 6

Where:

\tilde{z}(x)=z(f⁻¹(x))∥τ·∇(f⁻¹(x))∥ Eq. 7

With τ the unit tangent vector of a ∂\tilde{Ω} and \tilde{γ} being the conductivity, defined as follows:

$$\overline{Y} ( x ) := {F}_{*} Y ( x ) = \frac{{F}^{1} ( y ) Y ( y ) {( {F}^{1} ( y ) )}^{T}}{\vert {det F}^{1} ( y ) \vert} y = {F}^{- 1} ( x ) \qquad Eq . 8$$

where F′=DF is the Jacobi matrix of map F, and F*γ is the push-forward of γ by F.

The boundary measurements transform is defined as follows:

$${( \tilde{R} h ) ( x ) = ( R ( h ∘ \int ) ) ( y ) \vert}_{y = {\int }_{ }^{- 1} ( x ) } \qquad Eq . 9$$

where \tilde{R} corresponds to the conductivity \tilde{γ} and contact impedance \tilde{z} in the domain \tilde{Ω}. It should be noted that Equation 8 implies that even if γ is isotropic, the transformed conductivity \tilde{γ} will in general be anisotropic. It should be noted that the Dirichlet-to-Neumann map, and accordingly the Robin-to Neumann map does not uniquely determine an anisotropic conductivity.

However, the quadratic form corresponding to the push-forward Robin-to-Neumann map Rₘ is:

$${R}_{m} [ g , g ] = {\int }_{\partial Ω m}^{ } {gR}_{m} gdS = {\int }_{\partial Ω}^{ } ( g ∘ {f}_{m} ) {R}_{ϒ} ( g ∘ {f}_{m} ) dS \qquad Eq . 10$$

where the following represents the power needed to maintain g∘fₘ on the original boundary ∂Ω:

h∈H^{-1/2}(∂Ωₘ) Eq. 11

It should be noted that knowing Rₘ is equivalent to knowing the corresponding quadratic form.

The various embodiments reconstruct a conductivity up to a conformal deformation close to the original conductivity by determining γ in Ωₘ, which is an anisotropic conductivity that is as close as possible to isotropic conductivities, and then finds the isothermal coordinates to determine a deformation that makes the conductivity isotropic.

Specifically, let the following be a matrix-valued conductivity:

|γ^{j,k}(x)|_{j,k=1}² Eq. 12

Equation 12 has elements in L^{∞}(Ω) and where λ₁(x) and λ₂(x), with λ₁(x) less than or equal to λ₂(x), are the eigenvalues of γ^{jk}(x). The maximal anisotropy of a conductivity is A(γ) defined as:

$$A ( γ ) {=}_{x ∈ Ω}^{sup A ( γ , x ) ,} , where A ( γ , X ) = \frac{\sqrt{λ ( x )} - 1}{\sqrt{λ ( x )} + 1} , λ ( x ) = \frac{{λ}_{1} ( x )}{{λ}_{2} ( x )} \qquad Eq . 13$$

The function A(γ,x) is the anisotropy of γ at x.

It should be noted that if F is K-quasiconformal and γ is an isotropic conductivity, then:

$$A ( F * γ ) = \frac{K - 1}{K + 1} \qquad Eq . 14$$

It also should be noted that among all anisotropic conductivities in the model domain Ωₘ with a given Dirichlet-to-Neumann map, or equivalently Rₘ, there is a unique conductivity γₑ that has the minimal anisotropy A(γₑ). The conductivity γₑ is of the form γₑ=\tilde{γ}_{λ,θ,η} and defined as follows:

$$\tilde{γ} λ , θ , η ( x ) = η ( x ) {θ ( x )}_{} ( \begin{matrix}{λ}^{1 / 2} & 0 \\ 0 & {λ}^{- 1 / 2}\end{matrix} ) {θ ( x )}_{- 1}^{} \qquad Eq . 15$$

where λ is greater than or equal to 1 and is a constant, η(x) [symbol] R₊ is a real-valued function with the following property:

η(x)=det(γ_{g}(x))^{1/2} Eq. 16

and R_{θ(x)} is a rotation matrix corresponding to angle θ(x) as follows:

$${θ}_{} = ( \begin{matrix}cos θ & sin θ \\ - sin θ & cos θ\end{matrix} ) \qquad Eq . 17$$

It should be noted that for the conductivities \tilde{γ}=\tilde{γ}_{λ,θ,η}, the anisotropy A(\tilde{γ},x) is constant in x and defined as:

$$A ( \tilde{γ} , X ) = \frac{{λ}^{1 \text{/} 2} - 1}{{λ}^{1 \text{/} 2} + 1} \qquad Eq . 18$$

Thus, such conductivities \tilde{γ} are uniformly anisotropic conductivities. It should be noted that there is a unique map Fₑ: Ω→Ωₘ such that Fₑ|∂_{Ω}=fₘ and γₑ=(Fₑ)*γ, and the conductivity γₑ may be used to compute a conductivity in Ωₘ, which is a deformed image of the original conductivity γ defined in Ω, which results in:

det(γe(x))^{1/2}=γ(y), y=Fₑ⁻¹(x), x∈Ωₘ Eq. 19

It should be noted that Rₘ determines γₑ, but not the original domain Ω or the map Fₑ: Ω→Ωₘ, which is the external quasiconformal map with boundary value fₘ.

Next, with a given Rₘ, the conductivity γₑ can be determined as the unique solution of the following minimization problem:

$$min {λ}_{1} \\ ( λ , θ , η ) ∈ s \\ S = \{ ( λ , θ , η ) ∈ \vert 1 , ∞ ) \times {L}^{∞} ( {Ω}_{m} ) \times {L}^{∞} ( {Ω}_{m} ) \vert {γ ( λ , θ , η )}_{} = {m}_{} \} \qquad Eq . 20$$

It should be noted that for noisy measurement data, Equation 20 can be approximated with the following regularized minimization problem:

$${min}_{( λ , θ , η )} {‖ {R}_{γ ( ( λ , θ , η )} - {R}_{m} ‖}_{L ( {H}^{- 1 / 2 ( \partial {Ω}_{m} )} \cdot {H}^{- 1 / 2 ( \partial {Ω}_{m} ) )}}^{2} + {ɛ}_{1 f ( λ ) +} {ɛ}_{2} {‖ θ ‖}_{{H}^{1} ( {Ω}_{m} )}^{2} + {ɛ}_{2} {‖ η ‖}_{{H}^{1} ( {Ω}_{m} )}^{2} \qquad Eq . 21$$

where f: [1,∞)→R₊ is a convex function that has a minimum near λ=1 and lim_{t→1}f(t)=lim_{t→∞}f(t)=∞ and ∈₁, ∈₂,∈₃>0 are the regularization parameters.

The isotropization at 72 will now be described in more detail. In particular, the Equation 20 and the approximation thereof in Equation 21 are extended by transforming the reconstructed conductivity γₑ to an isotropic conductivity. Specifically, γₑ is extended by zero to the whole C=R² and defining Fᵢ: C→C to be the unique solution of the problem, as follows:

∂̅Fᵢ(z)=μ(z)∂F_{i(z)}, z∈ [symbol] ,

Fᵢ(z)=z+h(z),

h(z)→0 as z→∞. Eqs. 22-24

Where

$$μ ( z ) = \frac{{γ}_{e , 11} - {γ}_{e , 22} + 2 {i}_{{γ}_{e , 12}}}{{γ}_{e , 11} + {γ}_{e , 22} + 2 \sqrt{{det}_{γ e}}} , γ e = {[ γ e , jk ( z ) ]}_{j , k = 1}^{2} \qquad Eq . 25$$

It should be noted that Equation 21 has a unique solution as:

|μ(z)|≦c₀<1 Eq. 26

where μ(z) disappears outside of Ωₘ. It also should be noted that the map z→Fᵢ(z) may be considered as the isothermal coordinates in which γₑ can be represented as an isotropic conductivity. Thus, the conductivity γₑ is isotropized by defining γᵢ as follows (according to Equation 8):

γᵢ(x)=(Fᵢ)*γe Eq. 27

The conductivity γᵢ is isotropic and may be defined as:

γᵢ(x)=(det_{γe})^{1/2}·Fᵢ⁻¹(x)=γ·(Fᵢ·Fₑ)⁻¹(x), x∈Ωᵢ=Fᵢ(Ωₘ) Eq. 28

where:

G=Fᵢ·Fₑ:Ω→Fᵢ(Ωₘ) Eq. 29

Thus, solving Equations 22-24, a coordinate transformation may be determined that recovers the shape of the object. As should be appreciated the isotropization is a single step solution with no iterations. It should be noted that in some embodiments, for example, when finding transducer or electrode locations, the locations may expressed as coordinates without changing the transformations.

Accordingly, in various embodiments Ω is defined as a bounded, simply corrected C^{1,α} domain with α>0. Then, assume that γ [symbol] L^{∞}(Ω) is an isotropic conductivity and R_{γ} the Robin-to-Neumann map. Also, let Ωₘ be a model of the domain satisfying the same regularity assumptions as Ω and fₘ: ∂Ω→∂Ωₘ be a C^{1,α} smooth orientation preserving diffeomorphism. Then, assume that a ∂Ωₘ is known and Rₘ=(fₘ)*R_{γ}. Then, let γₑ be the solution of the minimization problem defined in Equation 20, Fᵢ be the solution of Equations 22-24 and γᵢ=(Fᵢ)*γₑ. Then, the above results in:

γᵢ(x)=γ(G⁻¹(x)), x∈Ωᵢ=Fᵢ(Ωᵢ) Eq. 30

where G: Ω→Ωᵢ is a conformal map.

Thus, with respect to performing shape-deforming reconstruction at 74, the determined conductivity γᵢ can be considered as a conformally deformed image of the conductivity γ. As the map Fₑ corresponds to the minimally anisotropic conductivity and the maps Fᵢ and G are related to the minimally anisotropic conductivity, the deformation G determined above is small if Fₑ: Ω→Ωₘ is close to identity.

More particularly, the regularized minimization problem defined in Equation 21 is solved. It should be noted that the uniformly anisotropic conductivities as defined in Equation 15 have the following property:

\tilde{γ}λ,η,θ=\tilde{γ}λ′,η,θ′, Eq. 31

Where

$${λ}^{′} = \frac{1}{λ} and {θ}^{′} ( x ) = θ ( x ) + π / 2$$

Equation 21 can be reparameterized such that λ has values of λ>0. Thus, the discretized version of Equation 21 is used for finding the minimizer of:

F(n,θ,λ)=∥V−U((n,θ,λ)∥²+W_{η}(η)+W_{θ}(θ)+W_{λ}, η>0,λ>0 Eq. 32

where parameters η, θ, λ define uniformly anisotropic conductivity of the form in Equation 15 in Ωₘ and the regularizing penalty functions are defined as follows:

$${W}_{η} ( η ) = {∝}_{θ} {\sum }_{k = 1}^{m} {η}_{k}^{2} + {∝}_{1} {\sum }_{K = 1}^{M} {\sum }_{i ∈ 𝒩 K}^{ } {\vert {η}_{k} - {η}_{j} \vert}^{2} , \\ {W}_{θ} ( θ ) = {β}_{θ} {\sum }_{k = 1}^{m} {θ}_{k}^{2} + {β}_{1} {\sum }_{K = 1}^{M} {\sum }_{i ∈ 𝒩 K}^{ } {\vert {e}^{i {θ}_{k}} - {e}^{i {θ}_{j}} \vert}^{2} , \\ {W}_{λ} ( λ ) = {β}_{2} ( log ( λ ) + {v}^{- 2} {log ( λ )}^{2} ) , \qquad Eqs . 33 - 35$$

where α₀, α₁, β₀, β₁, β₂ are non-negative scalar valued regularization parameters and Nₖ denotes the 4-point nearest neighborhood system for pixel k in the pixel grid. To provide the positivity constraint for η and λ in the minimization problem, the minimization is performed with respect to the following parameterization:

(ξ,θ,ζ),ξ=log(η)∈ [symbol] ^{M},θ∈ [symbol] ^{M}, ζ=log(λ)∈ [symbol] Eq. 36

Thereafter, the constrained problem defined by Equation 32 is transformed into an unconstrained problem as follows:

F(ξ,θζ)=∥V−U(exp(ζ),θ,exp(ζ))∥²+W_{η}(exp(ξ))+W_{θ}(θ)+W_{λ}(exp(ζ)) Eq. 37

Equation 37 is solved using, for example, any suitable gradient based optimization technique. For example, the minimization of Equation 37 may be provided with a Gauss-Newton optimization method having an explicit line search algorithm.

The following estimates define the estimated uniformly anisotropic conductivity in the model domain Ωₘ:

η=exp(ξ),θ,λ=exp(ζ)

Then, in one embodiment, isotropization of the anisotropic conductivity is provided. In particular, the mapping for Fᵢ(z) is solved using Equations 22-25 as described below.

First, the solid Cauchy transform is defined by:

$$Pf ( z ) = - \frac{1}{π} {\int }_{c}^{ } \frac{f ( λ )}{λ - z} dm ( λ ) \qquad Eq . 38$$

The Beurling transform is defined by:

Sf=∂Pf Eq. 39

It should be noted that P is the inverse operator of ∂̅ and S transforms ∂̅ derivatives into ∂ derivatives as follows: S(∂̅f)=∂f. The Beurling transform can then be defined as a principal value integral as follows:

$$Sf ( z ) = - \frac{1}{π} {\int }_{c}^{ } \frac{f ( w )}{( w - z )} dw \qquad Eq . 40$$

Thereafter, substituting Equation 23 to Equation 22 result in:

∂̅(z)=μ(z)+μ(z)∂h(z)=μ(z)+μ(z)S(∂̅h)(z) Eq. 41

which can be written in the following form:

h(z)=P[I−μS]⁻¹μ(z) Eq. 42

The inverse operator in Equation 42 can be expressed as a convergent Neumann series based on |μ(z)|<1.

A periodic version of Equation 42 may be defined as follows. In particular, let R>0 be large such that:

supp(μ)⊂Ω̅ₘ⊂B(0,R)

Then, set ∈>0 and s=2R+3∈ and define a square Q:=[−s,s)². Then, a smooth cutoff function is defined as follows:

$$η ∈ {C}_{0}^{∞} ( {ℝ}^{2} ) , η ( z ) = \{ \begin{matrix}1 for \vert z \vert < 2 R + {ε}_{ζ} \\ 0 for \vert z \vert < 2 R + 2 ε\end{matrix} \qquad Eq . 43$$

Then define a 2-s periodic approximate Green's function \tilde{g} by setting the function to η(z)/(πz) inside Q and extending periodically as follows:

$$\tilde{g} ( z + j 2 s + il 2 s ) = \frac{η ( z )}{π2} for z ∈ Q \backslash 0 , j , l , ∈ \qquad Eq . 44$$

Equation 38 can then be viewed as a convolution on the plane with the non-periodic Green's function 1/(πz). The periodic approximate Cauchy transform is then defined as follows:

$$\tilde{P} f ( z ) = ( \tilde{g} \tilde{*} f ) ( z ) = {\int }_{Q}^{ } \tilde{g} ( z - w ) f ( w ) dw \qquad Eq . 45$$

where *̅ denotes the convolution on the torus.

Further, an approximate Beurling transform is defined in the periodic context as follows:

$$\tilde{β} ( z + j 2 s + il 2 s ) = \frac{η ( z )}{π {z}^{2}} for z ∈ Q \backslash 0 , j , l ∈ \qquad Eq . 46$$

Then, analogously to Equation 40, the following is set:

$$\tilde{S} f ( z ) := ( \tilde{β} \tilde{*} f ) ( z ) = {\int }_{Q}^{ } \tilde{β} ( z - w ) f ( w ) dw \qquad Eq . 47$$

Thus, the periodic version of Equation 41 is defined as:

\tilde{h}(z)−\tilde{P}[I−\tilde{μ}\tilde{S}]⁻¹\tilde{μ}(z) Eq. 48

where \tilde{μ} the periodic expression of μ.

Equation 48 (unlike Equation 41) is the finite computational domain of Equation 41, allowing a numerical evaluation. In particular, let φ be a function of supp(φ)⊂B(0,R) and denote \tilde{φ} the periodic extension of φ. Because the functions (πξ)⁻¹ and \tilde{g}(ξ) coincide for |ξ|=|z−w|<2R+∈, the following identity may be defined for |z|<∈:

$$( P ϕ ) ( z ) = \frac{1}{π} {\int }_{𝔻}^{ } \frac{ϕ ( w )}{z - w} dw = {\int }_{Q}^{ } \tilde{g} ( z - w ) \tilde{ϕ} ( w ) dw = ( \tilde{P} \tilde{ϕ} ) ( z ) \qquad Eq . 49$$

Equations 42 and 48 may be written using the Neumann series as follows:

\tilde{h}=\tilde{P}(\tilde{μ}+\tilde{μ}\tilde{S}(\tilde{μ})+\tilde{μ}\tilde{S}(\tilde{μ}\tilde{S}(\tilde{μ}))+\tilde{μ}\tilde{S}(\tilde{μ}\tilde{S}(\tilde{μ}\tilde{S}(\tilde{μ})))+ . . . ) Eqs. 50 and 51

Because μ is supported in B(0,R), a combination of Equations 49, 50 and 51 results in the following:

\tilde{h}(z)=h(z) for |z|<R Eq. 52

Thus, the function h(z₀) at any point zₒ [symbol] C may be evaluated approximately as follows:

1. Evaluate the function \tilde{h}(z) approximately for a fine grid of points z [symbol] B(0,R) by truncating the infinite sum defined in Equation 51. The numerical implementation of the operators \tilde{P} and \tilde{S} are described below.

2. If z₀ [symbol] B(0,R), then by Equation 52, h(z₀)=\tilde{h}(z₀), which can be interpolated.

3. If z₀∉B(0,R), then by Equation 52:

h(z₀)=P((∂̅\tilde{h})|_{B(0,R)})(z₀)

It should be noted that the ∂̅ derivative can be approximated by finite differences, and the non-periodic Cauchy transform P may be implemented using the numerical quadrature in Equation 38.

With respect to \tilde{P} and \tilde{S}, a positive integer m may be selected, with M=2^{m}, and h=2s/M. Then a grid Gₘ⊂Q may be defined as follows:

[symbol]

={jh|j∈ [symbol] ₘ²},

[symbol]

ₘ²={j=(j₁,j₂)∈ [symbol] ²|−2^{m-1}≦jₗ<2^{m-1},l=1,2} Eq. 53

It should be noted that the number of points in Gₘ is M².

Then, the grid approximation is defined as:

φₕ: [symbol] ₘ²→ [symbol] of a function φ: Q→ [symbol] by φₕ(j)=φ(jh)

And setting:

$${\tilde{ℊ}}_{h} ( j ) = \{ \begin{matrix}\tilde{ℊ} ( jh ) , & for j ∈ {Z}_{m}^{2} \backslash 0 \\ 0 , & for j = 0 ;\end{matrix} \qquad Eq . 54$$

It should be noted that jhεR² is interpreted as the complex number hjᵢ+ihj2. Thus, \tilde{g}ₕ is a M×M matrix with complex entries. Given a periodic function φ, the transform \tilde{P}φ is approximately defined as follows:

(\tilde{P}φₕ)ₕ=h² [symbol] ⁻¹( [symbol] (\tilde{g}ₕ)· [symbol] (φₕ)) Eq. 55

where [symbol] represents the discrete Fourier transform and denotes · an element-wise matrix multiplication. It should be noted that the convolution *̅ on the torus becomes multiplication under a Fast Fourier Transform.

The discrete Beurling transform is then given by:

(\tilde{S}φₕ)ₕ=h² [symbol] ⁻¹( [symbol] (\tilde{β}ₕ)· [symbol] (φₕ)) Eq. 56

where \tilde{β}ₕ is the complex-valued M×M matrix defined as:

$${\tilde{β}}_{h} ( j ) = \{ \begin{matrix}\tilde{β} ( jh ) , & for j ∈ {Z}_{m}^{2} \backslash 0 \\ 0 , & for j = 0 ;\end{matrix} \qquad Eq . 57$$

Accordingly, once the numerical approximations of the mapping Fᵢ(z)=z+h(z) are determined, the reconstruction of the original isotropic conductivity is reconstructed by determining the following: η(Re(Fᵢ(z),Im(Fᵢ(z))). As should be appreciated, the computation for this step is the interpolation of η(Re(Fᵢ(z),Im(Fᵢ(z))) from an irregular grid, which is given by the numerical solution of Fᵢ(z).

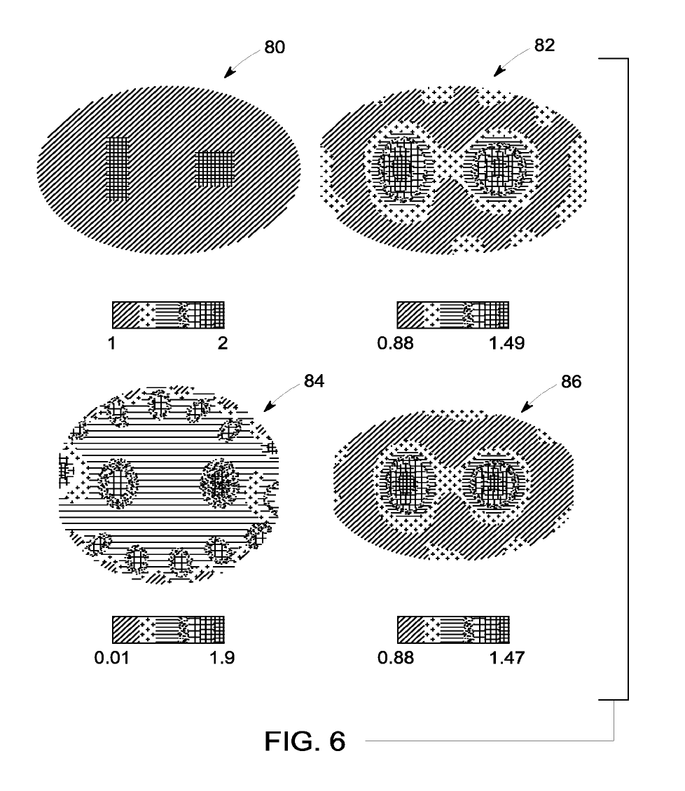

Thus, the various embodiments correct for the use of an incorrect model geometry. For example, as shown in FIG. 6, the image 80 illustrates a true conductivity and the measurement domain Ω. The image 82 illustrates a reconstruction of the isotopic conductivity using the correct domain Ω. The image 84 illustrates a reconstruction of isotropic conductivity using an incorrect model geometry Ωₘ without using the various embodiments. The image 86 illustrates reconstruction of isotropic conductivity using an incorrect model geometry Ωₘ, but using one or more of the embodiments described herein. As can be seen, the image 86 provides a more accurate reconstruction than the image 84. It should be noted that the image 86 shows the parameter η in the isothermal coordinates z→Fᵢ(z), namely the displayed quantity is η(Re(Fᵢ(z),Im(Fᵢ(z))).

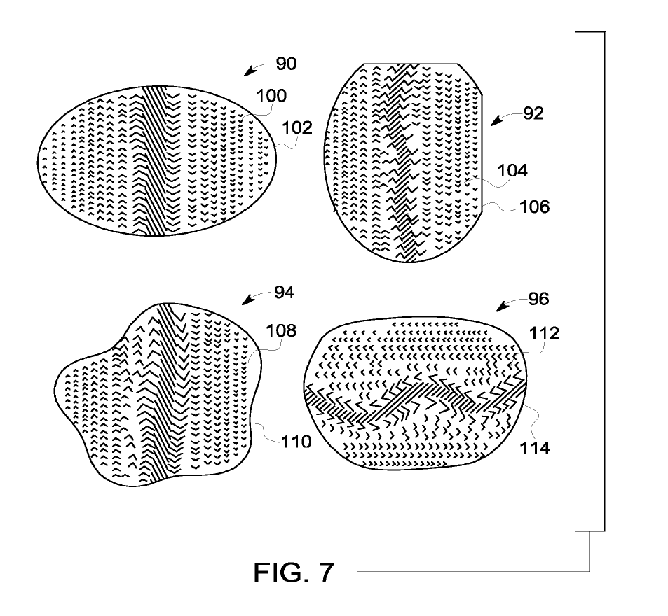

Additionally, FIG. 7 shows images 90, 92, 94 and 96 that correspond to the reconstruction wherein the measurement domain Ω is an ellipse, a truncated ellipse, a smooth curve and a segmented computerized tomography image of a human chest, respectively. The isothermal coordinates z→Fᵢ(z) are identified by the wavy lines 100, 104, 108 and 112, respectively, that were obtained from the numerical solution of Equations 22-25. The boundary (∂Ω) of the true measurement domain is shown by the solid lines 102, 106, 110 and 114, respectively. The images 90, 92, 94 and 96 show the approximate recovery of the deformation and domain boundary according to various embodiments.

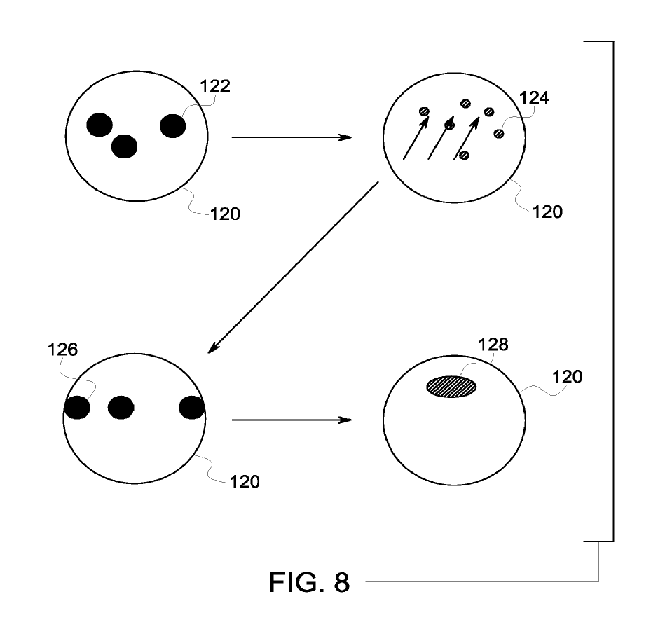

The various embodiments also may be used for two-dimensional reconstruction and to correct for electrode locations different than modeled. For example, as shown in FIG. 8, electrode locations 122 are in an initial assumed location within a boundary 120. Anisotropic impedance results and artifacts 124 within the boundary 120 are caused by an incorrect initial modeling of the location of the electrodes. Using the various embodiments, corrected electrode locations 126 are provided resulting in a correct isotropic impedance 128.

Thus, in various embodiments, a method is provided that 1) determines minimally anisotropic conductivity in the model domain that produces a measured data matrix, 2) performs isotropization of the anisotropic conductivity for finding the isothermal coordinates (approximate domain deformation) and 3) performs shape-deforming reconstruction.

The various embodiments and/or components, for example, the modules, elements, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as an optical disk drive, solid state disk drive (e.g., flash RAM), and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), field-programmable gate arrays (FPGAs), graphical processing units (GPUs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software”, “firmware” and “algorithm” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A soft-field tomography system comprising:
a plurality of transducers configured for positioning proximate a surface of an object to be imaged, the plurality of transducers configured to apply excitations to the surface of the object;
one or more excitation drivers coupled to the plurality of transducers and configured to generate excitation signals to be applied by the plurality of transducers;
one or more response detectors coupled to the plurality of transducers and configured to measure a response of the object at the plurality of transducers to the excitation signals applied by the plurality of transducers to acquire an Electrical Impedance Tomography (EIT) data set; and
a soft-field reconstruction module comprising at least one processor that executes a set of instructions stored in one or more storage elements, the soft-field reconstruction module configured to select a model domain for the EIT data set that represents a predetermined shape, determine a minimally anisotropic error in the model domain, and perform isotropization using the determined minimally anisotropic error to recover a boundary shape and isotropic conductivity.

2. The soft-field tomography system of claim 1, wherein the soft-field reconstruction module is further configured to determine the minimally anisotropic error by determining a minimally anisotropic conductivity in the model domain that reproduces measured EIT data corresponding to the EIT data set.

3. The soft-field tomography system of claim 1, wherein the soft-field reconstruction module is further configured to determine numerically isothermal coordinates for recovery of the boundary shape.

4. The soft-field tomography system of claim 3, wherein the soft-field reconstruction module is further configured to perform shape-deforming reconstruction using the isothermal coordinates to reconstruct an approximate original isotropic conductivity.

5. The soft-field tomography system of claim 1, wherein the object is a person and the system is further configured to monitor at least one of a heart function or a lung function of the person using the recovered boundary shape and isotropic conductivity for the EIT data set.

6. The soft-field tomography system of claim 1, wherein the model domain is a disc shape having approximately a same area as an actual boundary domain.

7. The soft-field tomography system of claim 1, wherein the soft-field reconstruction module is further configured to use a minimization algorithm and an approximation of the minimization algorithm to perform isotropization.

8. The soft-field tomography system of claim 1, wherein the soft-field reconstruction module is configured to reconstruct a conductivity, and to transform the reconstructed conductivity to the isotropic conductivity.

9. The soft-field tomography system of claim 8, wherein the following transformation is used to transform the reconstructed conductivity:
∂̅Fᵢ(z)=μ(z)∂F_{i(z)}, z∈ [symbol] ,
Fᵢ(z)=z+h(z),
h(z)→0 as z→∞.

10. The soft-field tomography system of claim 1, wherein the soft-field tomography system is configured to determine a coordinate transformation to recover the boundary shape.

11. The soft-field tomography system of claim 1, wherein the model domain comprises a model of an electrode.

12. The soft-field tomography system of claim 1, wherein the soft-field reconstruction module is configured to modify the model domain using a set of parameters that corrects for assumptions regarding shape of the object and electrode location on the object.
